(19) United States
(12) Patent Application Publication
(10) Pub. No.: US 2006/0182710 A1
(43) Pub. Date: Aug. 17, 2006

(54) Amphiphilic block copolymer and pharmaceutical formulation comprising the same

(75) Inventors: Ging-Ho Hsiue, Hsinchu (TW); Chau-Hui Wang, Kaohsiung City (TW); Wen-Hsiang Chang, Taipei City (TW); Chin-I Lin, Tainan County (TW); Shian-Jy Jassy Wang, Hsinchu County (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/314,009

(22) Filed: Dec. 22, 2005

(30) Foreign Application Priority Data
Dec. 31, 2004 (TW) 93141735

(51) Int. Cl. A61K 31/785

(52) U.S. Cl. 424/78.08; 424/78.27; 977/906

(57) ABSTRACT

An amphiphilic block copolymer and a pharmaceutical formulation comprising the same. The amphiphilic block copolymer comprises a poly(lactide) as a hydrophobic block and a poly(oxazoline) as a hydrophilic block.

### BACKGROUND

The invention relates to polymeric micelles, and more particularly, polymeric micelles comprising amphiphilic block copolymers.

Polymeric micelles appear to be one of the most advanced carriers for the delivery of drugs. The advantages of polymeric micelles include increased drug efficiency, low cellular toxicity, protection and stabilization of the drug, and targeting efficiency. In addition, the nano-sized micelles provide a prolonged circulation in the blood and also dodge mononuclear phagocyte system (MPS) and reticular endothelial system (RES). Therefore, polymeric micelles are the most attractive drug delivery system. In general, a micelle is an aggregate of amphiphilic copolymers. In an aqueous milieu, the hydrophobic compartment of an amphiphilic polymer has a tendency to self-assemble in order to avoid water and to minimize free interfacial energy of the system. A core-shell structure is formed and the hydrophobic inner core serves as a microenvironment for the solubilization of poorly water-soluble drugs. Previous studies also confirm that polymeric micelles carrying anti-cancer drugs effectively increase the stability and efficiency of the drugs.

Drugs encapsulated in nanoparticles must be stable and not leak in large amounts. A high release rate may lead to the precipitation of a hydrophobic drug in the blood vessels. In addition, low release rate is beneficial for the deposition of nanoparticles in appropriate tissues, improving local release. Conventional nanoparticles, however, release drugs by diffusion which is slow, and the diffusion site is not selective, thus, the effect of controlled release is reduced.

### SUMMARY

Accordingly, an amphiphilic block copolymer is provided, comprising a (poly(lactide)) (PLA) as a hydrophobic block, and a (poly(oxazoline)) (POz) as a hydrophilic block.

One embodiment of the amphiphilic block copolymer is as shown in formula (I):

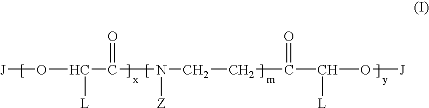

wherein

L represents H or C₁₋₆ alkyl;

Z represents H or C₂₋₂₁ acyl or phenyl;

J represents H or an organic residue derived from a ring-opening polymerization initiator; and

x, y, m represent an integer of 1 to 10,000.

Another embodiment of the amphiphilic block copolymer is as shown in formula (II):

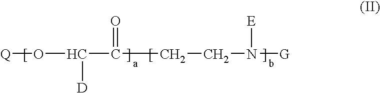

wherein

D represents H or C₁₋₆ alkyl;

E represents H or C2-21 acyl or phenyl;

G represents a residue derived from a nucleophilic reagent;

Q represents H or an organic residue derived from a ring-opening polymerization initiator; and

a, b represent an integer of 1 to 10,000.

A pharmaceutical formulation is also provided, comprising a micelle composed of the amphiphilic block copolymer, and a bioactive agent encapsulated in the micelle. In a neutral aqueous milieu, the micelle maintains its core-shell structure in which the hydrophilic blocks form the outer hydrophilic compartment and the hydrophobic blocks form the core compartment to encapsulate the bioactive agent. When the environmental pH value changes, for example, the pH value decreases, the micelles aggregate and the core-shell structure is destroyed to release the bioactive agent from the micelles.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

Amphiphilic block copolymer and pharmaceutical formulation comprising the same can be more fully understood and further advantages become apparent when reference is made to the following description and the accompanying drawings in which:

FIG. 1 is a diagram showing the synthesis of poly(L-lactide)-b-poly(2-ethyl-2-oxazoline)-b-poly(L-lactide) (PLLA-PEOZ-PLLA) in one embodiment of the amphiphilic block copolymer.

FIG. 2 is a diagram showing the deformation of PLLA-PEOZ-PLLA in the embodiment of the amphiphilic block copolymer. The left side shows a flower-like micelle before deformation at pH 7.4, and the right side shows aggregation of micelles after deformation at pH 5.1, PEOZ is outside; 2, PLLA is outside.

FIG. 3 is a diagram showing the process of intracellular drug delivery of PLLA-PEOZ-PLLA in the embodiment of amphiphilic block copolymer. 3, micelles; 4, cytoplasm; 5, endocytosis process; 6, endosome; 7, lysosome; 8, drugs are protected in the inner-core as they circulate in the blood; 9, drugs are released within acidic organelles; 10, outside cell and early endosome; 11, in endosome and lysosome.

FIG. 4 is a diagram showing the relationship between particle size and DP_{PLLA}. FIGS. 5A and 5B are diagrams showing the relationship between particle size and size distribution before (5A) and after (5B) doxorubicin (DOX) loading. FIG. 5A shows the average particle size before doxorubicin loading is 40 nm; FIG. 5B shows the average particle size after doxorubicin loading is 68 nm.

FIG. 6 is a diagram showing the I₁/I₃ intensity ratio from pyrene emission spectra of the PLLA-PEOz-PLLA micelles as a function of solution pH.

FIG. 7 is a diagram showing the results of in vitro release of doxorubicin in acidic and neutral aqueous milieu. The curve of solid squares indicates doxorubicin was fast released due to the deformation of micelles at pH 5.0; and the curve of open circles indicates micelles were stable in physiological condition over 7 days at pH 7.4.

FIG. 8 is diagram showing the results of in vitro cytotoxicity of the embodiment of the micelles. The Vertical axis indicates cell viability (%), the traverse axis indicates concentration (mg/mL). Solid squares indicate PLLA-PEOz-PLLA; solid triangles indicate positive control, branched polyethylenimine (B-PEI).

FIGS. 9A and 9B are the diagrams showing the results of cell growth inhibition by doxorubicin (DOX) or DOX-loaded micelles for one-day and three-days of coincubation, respectively. Solid squares indicate treatment of DOX-loaded micelles, and solid circles indicate treatment of free doxorubicin.

FIG. 10 represents the internalization and localization of free DOX and DOX-loaded micelles on HeLa cells observed by confocal laser scanning microscope (CLSM). (A) free DOX for 1 h of incubation, (B) micelles for 1 h of incubation, (C) micelles for 3 h of incubation, (D) micelles for 6 h of incubation, (E) micelles for 9 h of incubation. The red-fluorescence and green-fluorescence represent the localization of DOX and acidic organelles, respectively. (i.e. FIGS. 10A-1, 10B-1, 10C-1, 10D-1, and 10E-1 are red-fluorescence; FIGS. 10A-2, 10B-2, 10C-2, 10D-2, and 10E-2 are green-fluorescence; and 10A-3, 10B-3, 10C-3, 10D-3, and 10E-3 are overlaid images.)

### DETAILED DESCRIPTION

An amphiphilic block copolymer and a pharmaceutical formulation comprising the same are provided.

pH-responsive polymeric micelles have attracted considerable interest for several years, however, most studies focus on the physical properties of micelles rather than their application in controlled release of active drugs and other therapeutic compounds. The polymeric micelles were designed to have polyelectrolytes which alter aggregate morphologies of micelles in response to the dissociation status in aqueous solution. Supramolecular studies relating polystyrene-b-poly(acrylic acid) (PS-PAA) by A. Eisenberg et al. published in Science, 268, 1728(1995); and Science, 272, 1777(1996) reveal that PS-PAA has multiple morphologies at different pH milieu or in different salt concentrations, including spheres, rods, vesicles, and large compound vesicles (LCVs). The morphological changes are governed by the repulsion produced by the dissociation of PAA, or the interaction of ion bonds or bridging formed by Na⁺ or Ca²⁺ to PAA.

pH-responsive poly[4-vinylbenzoic acid-b-2-(N-morpholino)ethyl methacrylate (PVBA-PMEMA) zwitterionic diblock copolymer published by S. P. Armes is an example of the reversion of micelles in which the hydrophilicity and hydrophobicity of blocks are shifted in response to the dissociation of polyelectrolytes (Langmuir, 19, 4432(2003)). PVBA dissociates at high pH while PMEMA dissociates at low pH. The dissociated polymer has higher hydrophilicity, therefore, PVBA-core micelles form in acidic milieu and PMEMA-core micelles form in basic milieu. The micellar morphology of block copolymers can be reversed in certain conditions by salt-out. For example, poly[2-dimethylamino]ethyl methacrylate (PDEA) and poly[2-(N-morpholino)ethyl methacrylate] (PMEMA) have pKa of 7.3 and 4.9, respectively. Addition of Na₂SO₄ to PDEA-PMEMA biblock copolymer in aqueous phase at pH 6.7 leads to PMEMA-core micelle formation in response to the aggregation of PMEMA block (Macromolecules, 34, 1503(2001)).

In addition to diblock copolymers, triblock copolymers can be designed as responsive micelles. Poly(acrylic acid)-b-polystyrene-b-poly(4-vinyl pyridine) (PAA-PS-P4VP) ABC triblock copolymer has hydrophobic PS in the middle with acidic, hydrophilic PAA and basic, hydrophilic P4VP at the two ends. In acidic milieu, repulsion produced by the dissociation of P4VP results in the formation of micelles with PAA- and PS-core. On the contrary, the micelles reverse while the pH value increases (J. Am. Chem. Soc., 125, 15059(2003)).

The above mentioned studies all focus on the physical properties, and no discussion relating to the biocompatibility of these block copolymers is disclosed.

Moreover, Kataoka et al. link adriamycin and amphiphilic block copolymer, PEG-p(Asp-Hyd), with an acid-labile hydrazone bond to form PEG-p(Asp-Hyd-ADR) block copolymer. In acidic milieu, the cleavage of the acid-labile hydrazone bonds releases adriamycin (Angrw. Chem. Int. Ed., 42, 4640 (2003)). Covalent bonding, however, provides only small amounts of drug loading. In addition, the cleavage of hydrazone bonds may not be as effective as expected. For example, the release rate of PEG-p(Asp-Hyd-ADR) was less than 30% after 72-hour exposure at pH 5.0.

Diblock copolymer of POz and poly(ethylene oxide) (PEO) (WO 0110934, Feb. 15 (2001)) can be applied in biomedical field. Polyethylenimine (PEI) formed by the hydrolysis of POz is a hydrophilic polymer. PEI linked to negatively charged DNA may have increased hydrophobicity and a complex with PEI/DNA-core and PEO-outer layer can be formed (Macromolecules, 33, 5841 (2000)). The complex is a kind of non-viral gene vectors. The copolymer, however, loses environmentally sensitive properties after hydrolysis and DNA-bonding.

The inventors designed an environment-sensitive copolymer as a smart drug carrier and proved that the copolymer can release drug in accordance with the environmental pH changes. It is, therefore, provided an amphiphilic block copolymer including a poly(lactide) as a hydrophobic block and a (poly(oxazoline)) as a hydrophilic block.

The amphiphilic block copolymer can be the structure of formula (I):

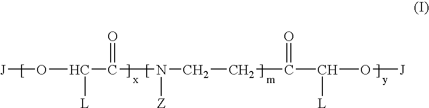

wherein

L represents H or C₁₋₆ alkyl;

Z represents H or C₂₋₂₁ acyl or phenyl;

J represents H or an organic residue derived from a ring-opening polymerization initiator; and

x, y, m represent an integer of 1 to 10,000.

Alternatively, the amphiphilic block copolymer can be the structure of formula (II):

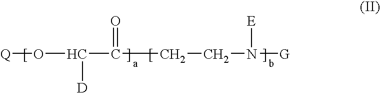

wherein

D represents H or C₁₋₆ alkyl;

E represents H, or C₂₋₂₁ acyl or phenyl;

G represents a residue derived from a nucleophilic reagent;

Q represents H or an organic residue derived from a ring-opening polymerization initiator; and

a, b represent an integer of 1 to 10,000.

The ratio of (x+y)/m in the amphiphilic block copolymer is preferably 0 to 5, more preferably 0 to 1.

“L” includes, but is not limited to, for example, methyl amino, carboxyl, or hydroxyl; preferably, methyl.

“z” includes, but is not limited to, for example, propionyl, acetyl, formyl, or carboxyl; preferably propionyl.

The ratio of a/b in the amphiphilic block copolymer is preferably 0 to 5, more preferably 0 to 1.

“D” includes, but is not limited to, for example, methyl, amino, carboxyl, or hydroxyl; preferably, methyl.

“E” includes, but is not limited to, for example, propionyl, acetyl, formyl, or carboxyl; preferably propionyl.

A pharmaceutical formulation is also provided, including a micelle composed of the aforementioned amphiphilic block copolymers, and a bioactive agent encapsulated therein. When the pH value of the milieu changes, for example, the pH value decreases, the bioactive agent can be released from the micelle. In addition, the particle size of the micelle is 10 nm to 1,000 nm in diameter; preferably, 20 nm to 200 nm in diameter. Moreover, the bioactive agent of the micelle is about 1 wt. % to about 60 wt. %; preferably, about 20 wt. % to about 40 wt. %.

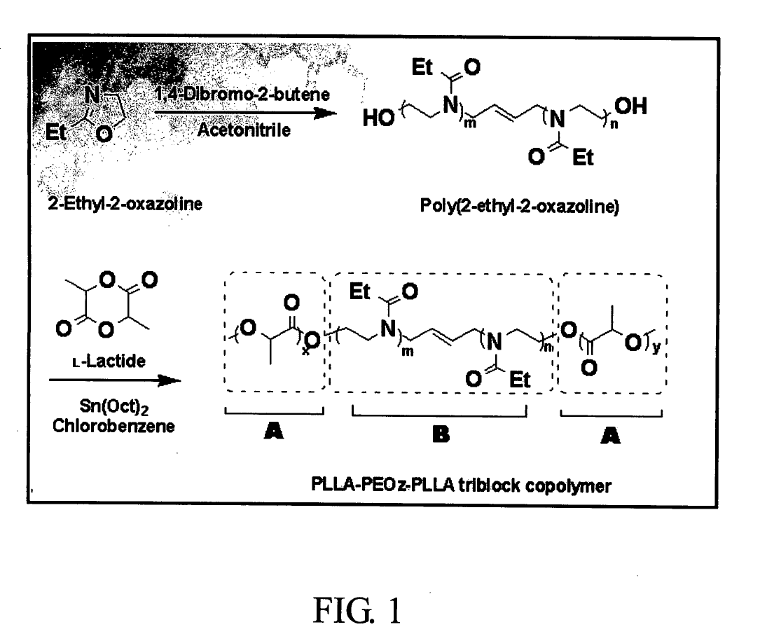

The micelle is composed of (poly(oxazoline)/poly(lactide) (POz/PLA). PLA is a hydrophobic and biodegradable polyester, and POz is a hydrophilic polymer. Both have low toxicity and good biocompatibility. In one embodiment of the preparation of the micelle, an example of the preparation of (poly(L-lactide)-b-poly(2-ehtyl-2-oxazoline)-b-poly(L-actide) (PLLA-PEOz-PLLA) is shown as FIG. 1. In the beginning, a bifunctional initiator, 1,4-dibromo-2-butene, was used for the cationic ring-opening polymerization of a monomer, 2-ethyl-2-oxazoline. After the polymerization, the reaction was cooled to room temperature, and KOH aqueous solution was added to introduce —OH into the two ends of PEOz. Next, L-lactide and HO-PEOz-OH were ring-opening polymerized under the presence of Sn(Oct)₂ as a catalyst. The obtained product, PLLA-PEOz-PLLA, was dried, collected, and frozen-stored.

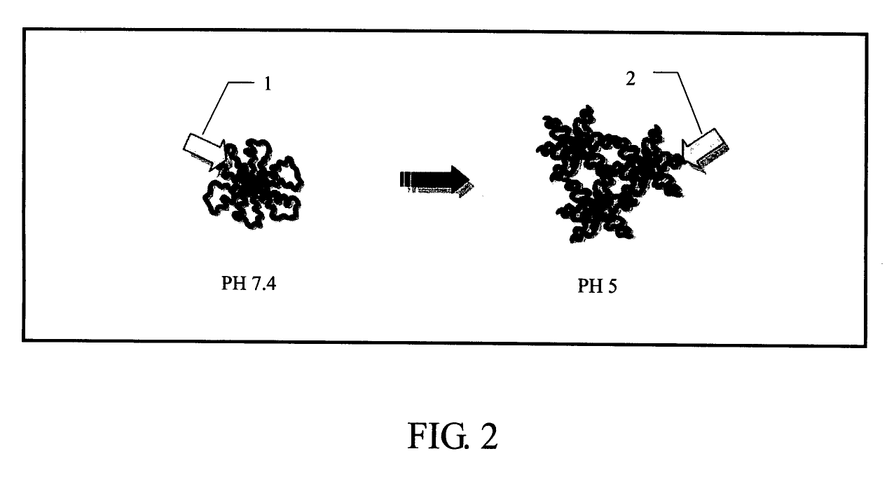

The formation of micelles of PLLA-PEOz-PLLA in response to environmental changes is shown in FIG. 2. Under neutral aqueous milieu (pH 7.4), the triblock copolymers form a core-shell structure in which hydrophilic PEOZ is outside (1) and hydrophobic PLLA is inside, providing a microenvironment for hydrophobic drugs, as the flower-like micelles shown on the left side of FIG. 2. When the pH value decreases in the aqueous solution (pH 5.0), PEOZ form intermolecular and intramolecular hydrogen bonds and aggregate, PLLA is extruded (2). The micelle structure is destroyed and the drugs are released, as shown in the right side of FIG. 2. This mechanism controls the release of drugs only in cancerous tissues, avoiding side effects of the anticancer drugs.

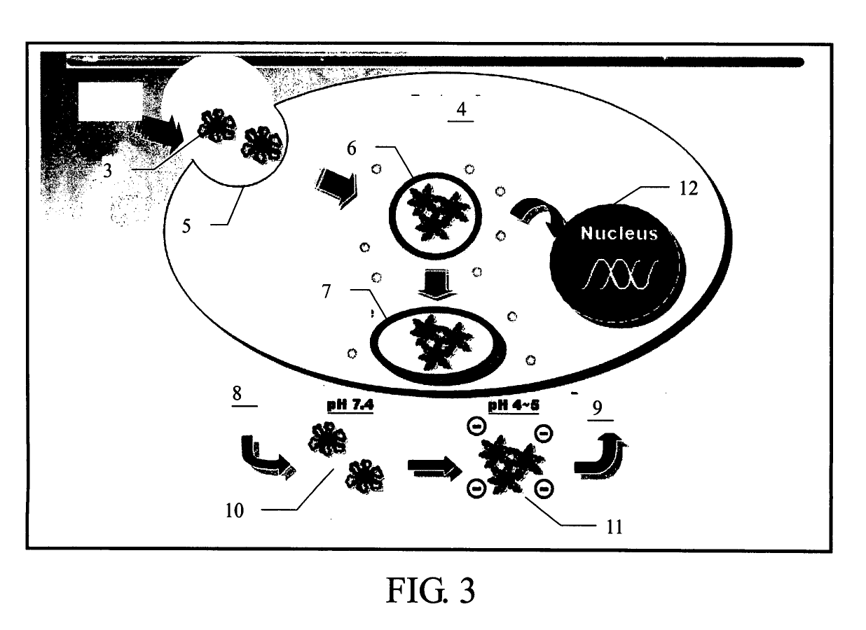

FIG. 3 shows the process of intracellular drug delivery of PLLA-PEOz-PLLA in the embodiment of amphiphilic block copolymer. Since enhanced permeation and retention effect (EPR) provides higher vascular permeability in cancerous tissues than in normal tissues, polymers with a larger molecular size can be permeated into cancerous tissues easily. In addition, the recovery of polymeric compounds by the lymph duct around the cancerous tissues is incomplete, and the cancerous tissues tend to retain polymeric compounds. Accordingly, micelle 3 containing anticancer drugs may enter a cancer cell by endocytosis 5. In the cytoplasm 4, the vesicle containing micelle 3 becomes an early endosome and then endosome 6. Drugs can be released by the decrease of environmental pH and entered nucleus 12. The endosome containing the remaining polymers finally becomes lysosome 7 and the polymers are metabolized. The underside picture of FIG. 3 shows the detailed mechanism. When micelles are outside cell or at the early endosome (10), the environmental pH maintains 7.4 and drugs are protected in the inner-core when circulating in the blood (8). When micelles are in the endosome or lysosome (11), the environmental pH decreases to 4˜5 and drugs are released within the acidic organelles (9).

Practical examples are described herein.

### EXAMPLES

### Example 1

### Preparation of PLLA-PEOz-PLLA formula

Four hundred twenty mg (2 mmol) of bifunctional initiator, 1,4-dibromo-2-butene, was added to a two-necked rounded bottom flask connecting to a cold finger and a sleeve stopper. The device connected to vacuum tube and dry nitrogen was introduced thereto. Sixty mL of acetonitrile was introduced. The reaction was controlled at 100° C. in oil bath. Ten mL of dehydrated monomer, 2-ethyl-2-oxazoline, (100 mmol) was then added and the reaction was performed under a recirculating system for 16 hours. The device was cooled down to room temperature at the end of the reaction. Then, 0.1 N of KOH aqueous solution was added and mixed for 1 hour to introduce —OH to the two ends of PEOZ. The product was dried and dissolved again in chloroform. Silica gel chromatography was performed to eliminate salts and impurities. The product was finally precipitated in diethyl ether twice. After drying at 40° C. for 24 hours, the purified product was identified as HO-PEOZ-OH with a yield of over 98%.

The purified L-lactide (582 mg) and HO-PEOz-OH (2 g) were placed in a two-necked rounded bottom flask connected to a cold finger and a vacuum tube. After drying at 50° C. for 30 min and nitrogen replacement for three times, 7.5 mL of chlorobenzene was introduced. Raising the temperature to 140° C. under a recirculation system, the reaction was performed for 16 hours in the presence of 1 wt. % of Sn(Oct)₂ as a catalyst. After the reaction, impurities were eliminated by silica gel chromatography with chlorobenzene as mobile phase. The product was precipitated by diethyl ether for three times. The obtained PLLA-PEOZ-PLLA triblock copolymer was dried at 40° C. for 24 hours and collected. The yield was>90%.

### Example 2

### Particle Size and Micelle Deformation Analysis of the Formulated PLLA-PEOz-PLLA

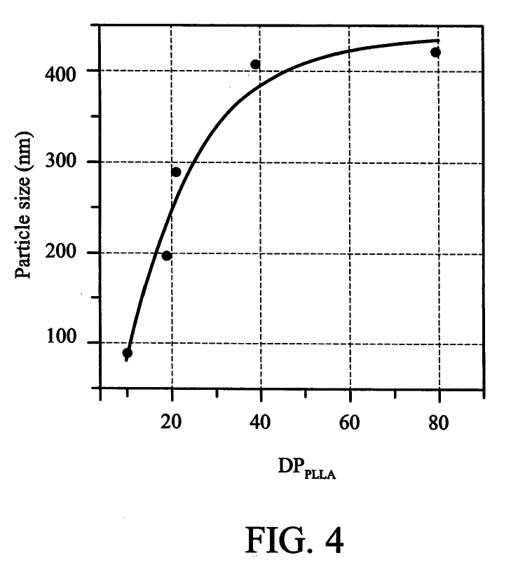

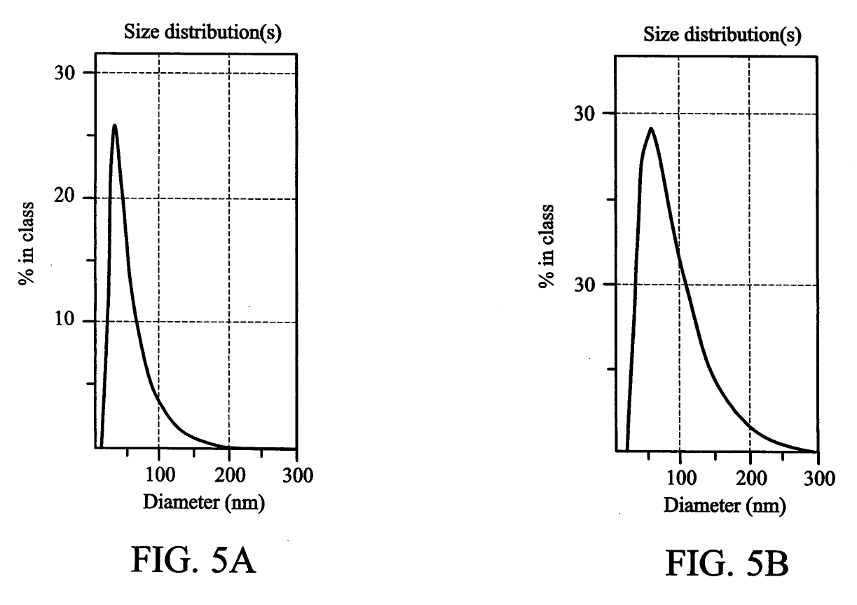

Different formulations of PLLA-PEOz-PLLA, including ABA-5K20, ABA-5K40, ABA-10K20, ABA-10K40, and ABA20K40 (A, B are as shown in FIG. 1), were placed in neutral solution (deionized water). The particle size of micelles produced by each formulation was measured by a particle size analyzer. The results shown as FIG. 4 proved that the particle size increases with the increase of PLLA chain length.

Micelles formulated by ABA5K20 (ABA ratio: 0.2) of PLLA-PEOz-PLLA were analyzed before and after doxorubicin loading by a particle size analyzer. The measurement was performed in DMSO at 485 nm. The results were shown in FIGS. 5A and 5B. Before loading, the average particle size of the micelles was about 40 nm, and it was about 68 nm after loading. The drug loading efficiency was about 31%. The calculation was by the formula of: (W_{DOX in micelles}/W_{DOX-loaded micelles})×100%. The appropriate particle size of micelles is usually between 20˜200 nm. The micelle with a particle size larger than 200 nm may be engulfed by macrophages, whereas the micelle with a particle size less than 20 nm may be metabolized. Therefore, the average particle size of the micelles in the example is suitable for drug delivery.

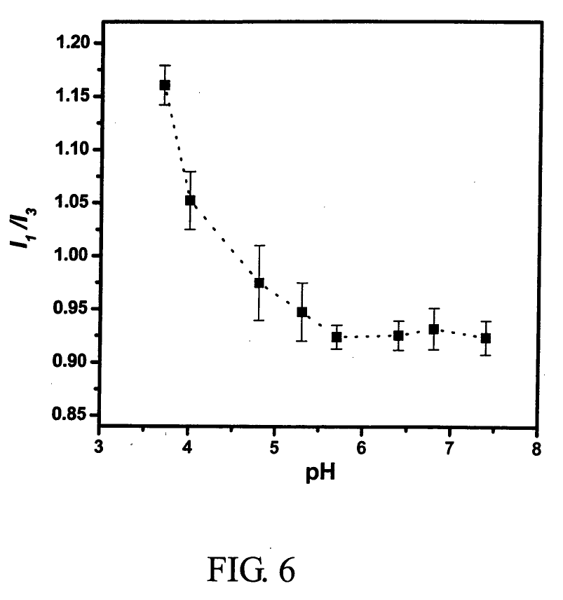

FIG. 6 illustrated the pH-induced deformation of micelles as determined by pyrene fluorescence studies. Pyrene is a highly hydrophobic chemical and therefore preferentially migrates into the hydrophobic micelle core in aqueous solution. The behavior of micelle deformation was observed from the increased intensity ratio (I₁/I₃), which indicated a more hydrophilic environment for the pyrene probe. At low pH, the micelle aggregated to expose PLLA outside, resulting in increased intensity ratio (I₁/I₃).

### Example 3

### Analysis of In Vitro Release and Cytotoxicity

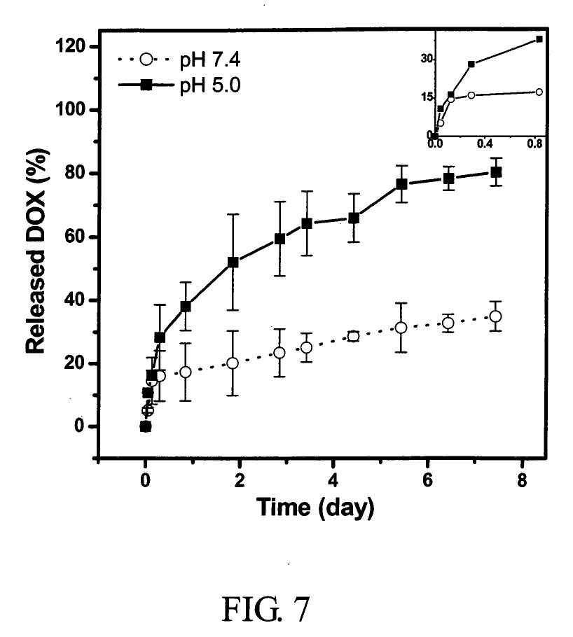

In vitro release of doxorubicin was measured in acidic and neutral aqueous milieu. Since doxorubicin appears red in color, it could be detected by UV/Vis spectrophotometer (Uv/Vis: Perkin Elmer Lamda 2S). The results were shown in FIG. 7. The curve of solid squares indicates that doxorubicin was released by the destruction of micelles at pH 5.0. The curve of open circles indicates that the micelles maintain stable over 7 days at pH 7.4, a physiological environment.

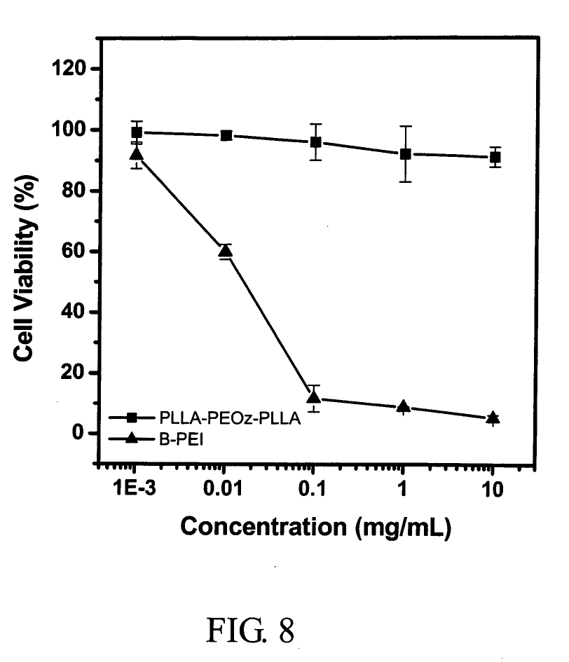

In vitro toxicity test is directed to cytotoxicity of micelles. Human cervical cancer cell line HeLa was applied. Micelle formulation of PLLA-PEOZ-PLLA (ABA-5K20) and branched polyethylenimine (B-PEI) as control at different concentrations were used. Cell viability was read in a 96-well plate by ELISA reader (Awareness Stat Fax 2100) with MTT assay. The results were shown in FIG. 8. Each point indicates six repeats. The results showed that PLLA-PEOZ-PLLA prepared in example 1 has low cytotoxicity whereas the control, B-PEI, has higher cytocoxicity. When PLLA-PEOz-PLLA was in a concentration up to 10 mg/mL, most cells were still alive. When B-PEI was in a concentration of 0.01 mg/mL, only 60% of the cells were survived.

### Example 4

### In Vitro Cancer Cell Growth Inhibition with Micelles Containing Anticancer Drug Doxorubicin

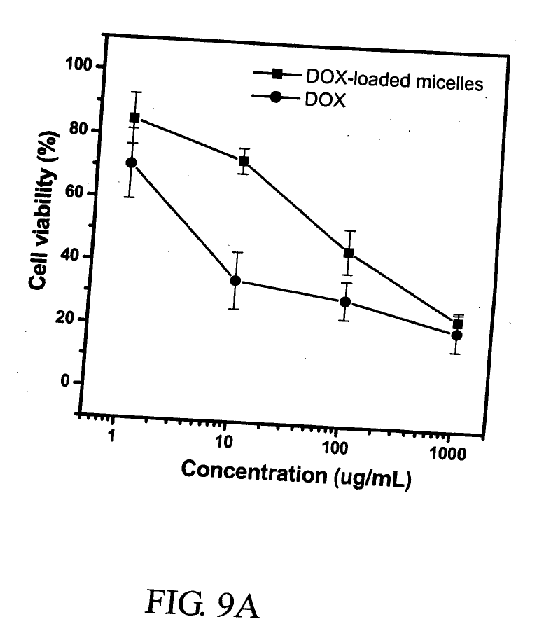

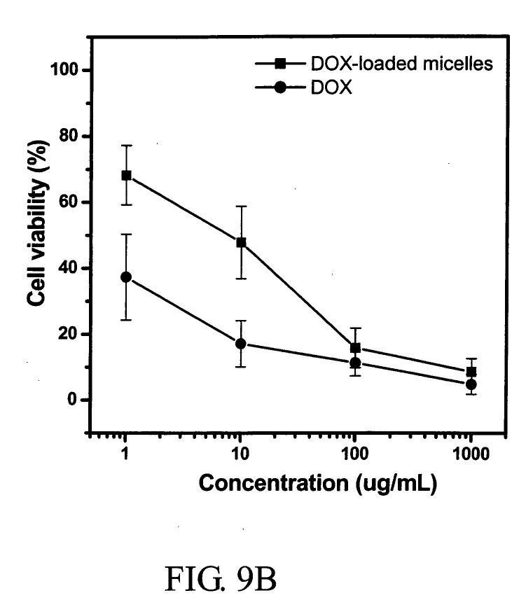

Growth inhibition of micelles containing doxorubicin or doxorubicin alone was examined in HeLa cell line. One ug/mL, 10 ug/mL, 100 ug/mL, and 1000 ug/mL of doxorubicin or micelles containing doxorubicin were applied to HeLa cells. Cell viability was measured after 24- or 72-hour treatment in a 96-well plate by ELISA reader (Awareness Stat Fax 2100) with MTT assay. The results were shown in FIG. 9. Each point indicates six repeats. The results showed that the growth of HeLa cells could be effectively inhibited after 72-hour treatment of micelles containing doxorubicin at a concentration of 0.1 mg/mL. The efficiency is equivalent to the direct application of doxorubicin.

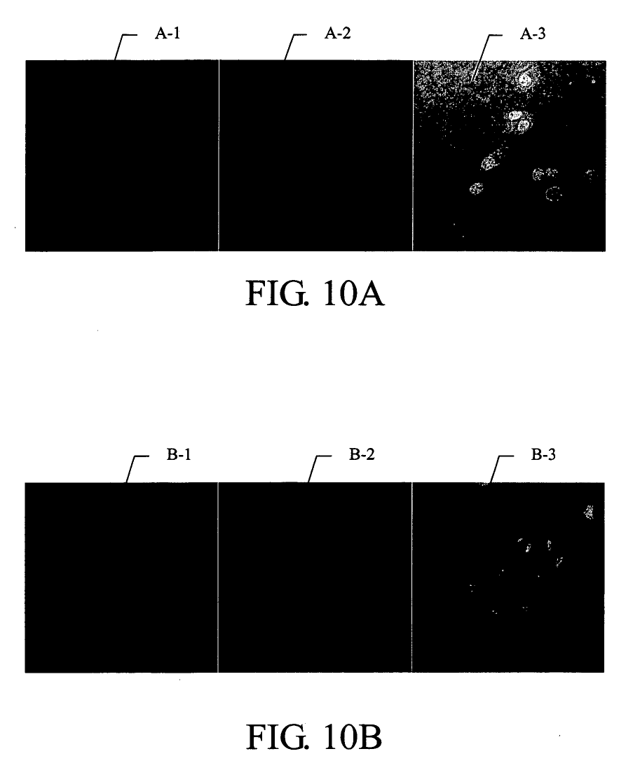

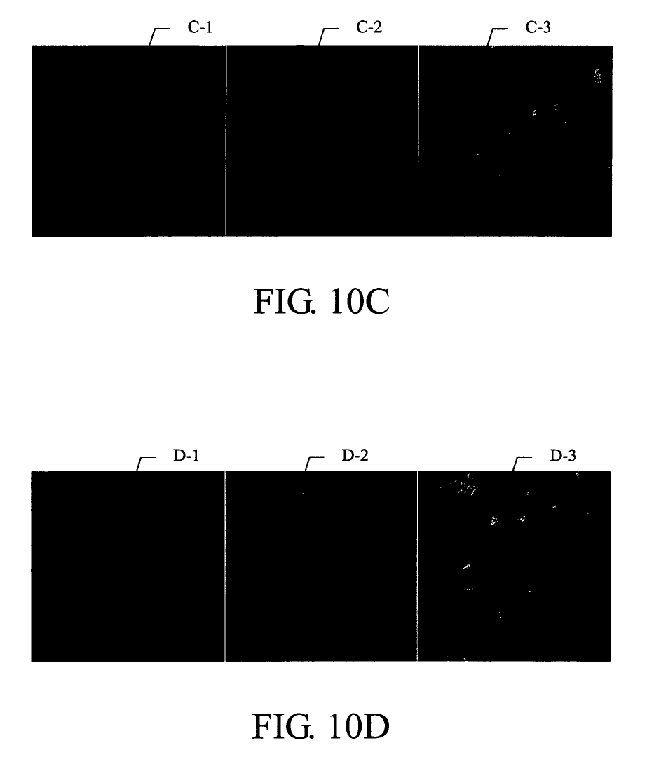

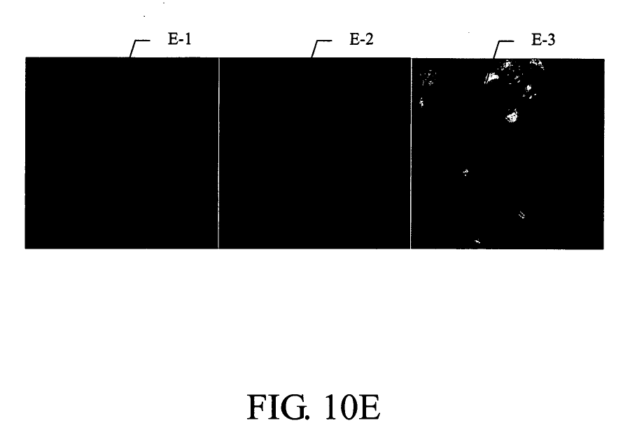

The endocytosis of micelles and the release of doxorubicin were observed under confocal laser scanning microscopy (CLSM: Zeiss) and phase contrast microscopy (Wild MPS 51S) and the results were shown in FIG. 10A˜10E. FIG. 10A are microscopic photographs showing HeLa cells after one-hour doxorubicin treatment. FIG. 10B˜10E are microscopic photographs showing HeLa cells treated with micelles containing doxorubicin for 1, 3, 6, 9 hours. Green fluorescence represents acidic organelles, and red fluorescence represents doxorubicin. The overlapping indicates that doxorubicin was successfully released from the acidic organelles.

While the invention has been described by way of example and in terms of preferred embodiment, it is to be understood that the invention is not limited thereto.

What is claimed is:

1. An amphiphilic block copolymer comprising a poly(lactide) as a hydrophobic block and a poly(oxazoline) as a hydrophilic block.

2. The amphiphilic block copolymer as claimed in claim 1, having the structure of formula (I): 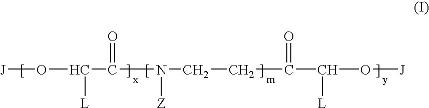
wherein
L represents H or C₁₋₆ alkyl;
Z represents H or C₂₋₂₁ acyl or phenyl;
J represents H or an organic residue derived from a ring-opening polymerization initiator; and
x, y, m represent an integer of 1 to 10,000.

3. The amphiphilic block copolymer as claimed in claim 2, wherein the ratio of (x+y)/m is 0 to 5.

4. The amphiphilic block copolymer as claimed in claim 2, wherein the ratio of (x+y)/m is 0 to 1.

5. The amphiphilic block copolymer as claimed in claim 2, wherein L is methyl, amino, carboxyl, or hydroxyl.

6. The amphiphilic block copolymer as claimed in claim 5, wherein L is methyl.

7. The amphiphilc block copolymer as claimed in claim 2, wherein Z is propionyl, acetyl, formyl, or carboxyl.

8. The amphiphilic block copolymer as claimed in claim 7, wherein Z is propionyl.

9. The amphiphilic block copolymer as claimed in claim 1, having the structure of formula (II): 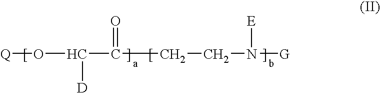
wherein
D represents H or C₁₋₆ alkyl;
E represents H, or C₂₋₂₁ acyl or phenyl;
G represents a residue derived from a nucleophilic reagent;
Q represents H or an organic residue derived from a ring-opening polymerization initiator; and
a, b represent an integer of 1 to 10,000.

10. The amphiphilic block copolymer as claimed in claim 9, wherein the ratio of a/b is 0 to 5.

11. The amphiphilic block copolymer as claimed in claim 9, wherein the ratio of a/b is 0 to 1.

12. The amphiphilic block copolymer as claimed in claim 9, wherein D is methyl, amino, carboxyl, or hydroxyl.

13. The amphiphilic block copolymer as claimed in claim 12, wherein D is methyl.

14. The amphiphilic block copolymer as claimed in claim 9, wherein E is propionyl, acetyl, formyl, or carboxyl.

15. The amphiphilic block copolymer as claimed in claim 14, wherein E is propionyl.

16. A pharmaceutical formulation, comprising:
a micelle composed of the amphiphilic block copolymers as claimed in claim 1; and
a bioactive agent encapsulated therein.

17. The pharmaceutical formulation as claimed in claim 16, wherein the amphiphilic block copolymer has the structure of formula (I): 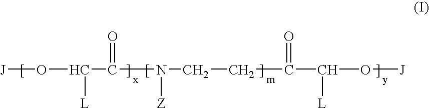
wherein
L represents H or C₁₋₆ alkyl;
Z represents H or C₂₋₂₁ acyl or phenyl;
J represents H or an organic residue derived from a ring-opening polymerization initiator; and
x, y, m represent an integer of 1 to 10,000.

18. The pharmaceutical formuation as claimed in claim 17, wherein the ratio of (x+y)/m is 0 to 5.

19. The pharmaceutical formulation as claimed in claim 17, wherein the ratio of (x+y)/m is 0 to 1.

20. The pharmaceutical formulation as claimed in claim 17, wherein L is methyl, amino, carboxyl, or hydroxyl.

21. The pharmaceutical formulation as claimed in claim 20, wherein L is methyl.

22. The pharmaceutical formulation as claimed in claim 17, wherein Z is propionyl, acetyl, formyl, or carboxyl.

23. The pharmaceutical formulation as claimed in claim 22, wherein Z is propionyl.

24. The pharmaceutical formulation as claimed in claim 17, the amphiphilic block copolymer has the structure of formula (II): 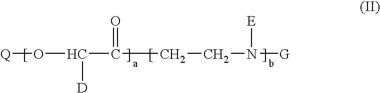
wherein
D represents H or C₁₋₆ alkyl;
E represents H, or C₂₋₂₁ acyl or phenyl;
G represents a residue derived from a nucleophilic reagent;
Q represents H or an organic residue derived from a ring-opening polymerization initiator; and
a, b represent an integer of 1 to 10,000.

25. The pharmaceutical formulation as claimed in claim 24, wherein the ratio of a/b is 0 to 5.

26. The pharmaceutical formulation as claimed in claim 24, wherein the ratio of a/b is 0 to 1.

27. The pharmaceutical formulation as claimed in claim 24, wherein D is methyl, amino, carboxyl, or hydroxyl.

28. The pharmaceutical formulation as claimed in claim 27, wherein D is methyl.

29. The pharmaceutical formulation as claimed in claim 24, wherein E is propionyl, acetyl, formyl, or carboxyl.

30. The pharmaceutical formulation as claimed in claim 29, wherein E is propionyl.

31. The pharmaceutical formulation as claimed in claim 16, wherein the bioactive is released from the micelle when the pH value of the milieu changes.

32. The pharmaceutical formulation as claimed in claim 16, wherein the micelle is 10 nm to 1,000 nm in diameter.

33. The pharmaceutical formulation as claimed in claim 32, wherein the micelle is 20 nm to 200 nm in diameter.

34. The pharmaceutical formulation as claimed in claim 16, wherein the bioactive agent encapsulated in the micelle is 1 wt. % to 60 wt. %.

35. The pharmaceutical formulation as claimed in claim 34, wherein the bioactive agent encapsulated in the micelle is 20 wt. % to 40 wt. %.
